(19) United States
(12) Patent Application Publication
(10) Pub. No.: US 2018/0338688 A1
(43) Pub. Date: Nov. 29, 2018

(54) MEASUREMENT APPARATUS, MEASUREMENT INSTRUMENT, MEASUREMENT SYSTEM, SERVER, ANALYSIS METHOD, STORAGE MEDIUM, AND DATA STRUCTURE

(71) Applicant: KYOCERA Corporation, Kyoto-shi (JP)

(72) Inventor: Susumu KASHIWASE, Machida-shi (JP)

(21) Appl. No.: 15/987,940

(22) Filed: May 24, 2018

(30) Foreign Application Priority Data
May 26, 2017 (JP) 2017-105025

(51) Int. Cl. A61B 5/01; G01K 13/00; A61B 5/00

(52) U.S. Cl. CPC A61B 5/01; G01K 13/004; A61B 2562/16; A61B 2562/0271; A61B 5/6817

(57) ABSTRACT

Provided are a measurement apparatus, a measurement instrument, a measurement system, a server, an analysis method, a storage medium, and a data structure that can easily output good sound while improving the accuracy of body temperature detection. The measurement apparatus includes a metal tube having a first end and a second end; a measurement unit which is arranged at the first end side of the metal tube and is capable of measuring electromagnetic radiation incident from the second end of the metal tube; a housing for holding the metal tube; and a vibrator for vibrating the housing.

### CROSS-REFERENCE TO RELATED APPLICATION

The present application claims priority to and the benefit of Japanese Patent Application No. 2017-105025 filed May 26, 2017, the entire contents of which are incorporated herein by reference.

### TECHNICAL FIELD

The present disclosure relates to a measurement apparatus, a measurement instrument, a measurement system, a server, an analysis method, a storage medium, and a data structure.

### BACKGROUND

Earphones capable of measuring body temperature are known. For example, a canal type earphone provided with a body temperature sensor is known.

### SUMMARY

A measurement apparatus according to a first aspect of the present disclosure includes a metal tube, a measurement unit, a housing, and a vibrator. The metal tube has a first end and a second end. The measurement unit is arranged at a first end side of the metal tube and is capable of measuring electromagnetic radiation incident from the second end of the metal tube. The housing is configured to hold the metal tube. The vibrator is configured to vibrate the housing.

A measurement instrument according to a second aspect of the present disclosure includes a first measurement unit configured as a blood flow measurement apparatus capable of measuring blood flow, and the measurement apparatus according to the first aspect.

A measurement system according to a third aspect of the present disclosure includes the measurement apparatus according to the first aspect; a control apparatus for controlling operation of the measurement apparatus; and a server connected to the measurement apparatus via a network, wherein the server is configured to receive information on electromagnetic radiation measured by the measurement apparatus, analyze the received information based on information stored in a memory, and transmit the analyzed information to the control apparatus.

A server according to a fourth aspect of the present disclosure is connected to a control apparatus for controlling operation of the measurement apparatus according to the first aspect via a network; wherein the server is configured to receive information on electromagnetic radiation measured by the measurement apparatus, analyze the received information based on information stored in a memory, and transmit analysis information indicating a result of the analysis to the control apparatus.

An analysis method according to a fifth aspect of the present disclosure is to be performed by a server connected to a control apparatus for controlling operation of the measurement apparatus according to the first aspect via a network; wherein the analysis method includes receiving information on electromagnetic radiation measured by the measurement apparatus, analyzing the received information based on information stored in a memory, and transmitting analysis information indicating a result of the analysis to the control apparatus.

A non-transitory computer readable storage medium storing an analysis program according to a sixth aspect of the present disclosure which, when executed by a computer, causes the computer to receive information on electromagnetic radiation measured by the measurement apparatus according to the first aspect, analyze the received information based on information stored in a memory, and transmit analysis information indicating a result of the analysis to a control apparatus.

A data structure according to a seventh aspect of the present disclosure is configured for use in a computer, including information on electromagnetic radiation measured by a measurement apparatus according to the first aspect, a user ID of a user of the measurement apparatus, an average body temperature of a group to which the user belongs, and an individual average body temperature of the user; wherein the computer is used to determine whether information on electromagnetic radiation measured by the measurement apparatus is abnormal based on at least one of the average body temperature and the individual average body temperature.

### BRIEF DESCRIPTION OF THE DRAWINGS

In the accompanying drawings:

FIG. 1 is a schematic diagram illustrating the internal structure of a measurement apparatus according to one embodiment of the present disclosure;

FIG. 2 illustrates an example of acoustic characteristics;

FIG. 3 is a schematic diagram illustrating the appearance of a measurement instrument provided with the measurement apparatus in FIG. 1;

FIG. 4 is a cross-sectional view taken from line A-A of the measurement instrument in FIG. 3;

FIG. 5 is a schematic diagram illustrating a connection configuration of the measurement instrument in FIG. 3 and a control apparatus;

FIG. 6 illustrates an example of functional blocks provided with the control apparatus in FIG. 5;

FIG. 7 is a conceptual diagram of sound data to be stored in a memory of the control apparatus in FIG. 5;

FIG. 8 is a schematic diagram illustrating a configuration example of a measurement system in which the measurement instrument in FIG. 5 is used;

FIG. 9 is a functional block diagram illustrating the schematic configuration of the server in FIG. 8;

FIG. 10 is a sequence chart illustrating an example of processing performed by the measurement system in FIG. 8;

FIGS. 11A, 11B, 11C, and 11D are configuration diagrams illustrating an example of data to be stored in the server in FIG. 8;

FIG. 12 is a flowchart illustrating an example of body temperature information analysis processing performed by the server in FIG. 8;

FIG. 13 illustrates a configuration example of a measurement system according to Embodiment 2;

FIG. 14 illustrates an example of functional blocks provided with the smartphone in FIG. 13;

FIG. 15 is a schematic diagram illustrating a display screen of the smartphone in FIG. 13;

FIG. 16 is a sequence chart illustrating an example of processing performed by the measurement system in FIG. 13; and

FIG. 17 is a schematic diagram illustrating the internal structure of a variation of the measurement apparatus.

### DETAILED DESCRIPTION

For conventionally known earphones capable of measuring body temperature, it is difficult to output good sound while improving the accuracy of body temperature detection.

For example, in a conventionally known earphone, a body temperature sensor is provided substantially in the center of an opening of an acoustic cylinder configured to face an eardrum. In this case, body temperature measured by the body temperature sensor may have absorbed infrared energy radiated from, for example, the wall surface of an ear canal or the like, other than the eardrum. The wall surface of the ear canal or the like, however, is more easily affected by outside air than the eardrum. Thus, when the body temperature sensor measures body temperature based on infrared energy radiated from the wall surface of an ear canal or the like, it is difficult to accurately measure the body temperature of a subject.

It would be helpful to provide a measurement apparatus, a measurement instrument, a measurement system, a server, an analysis method, a storage medium, and a data structure that can easily output good sound while improving the accuracy of body temperature detection.

A plurality of embodiments according to the present disclosure will be described with reference to the accompanying drawings.

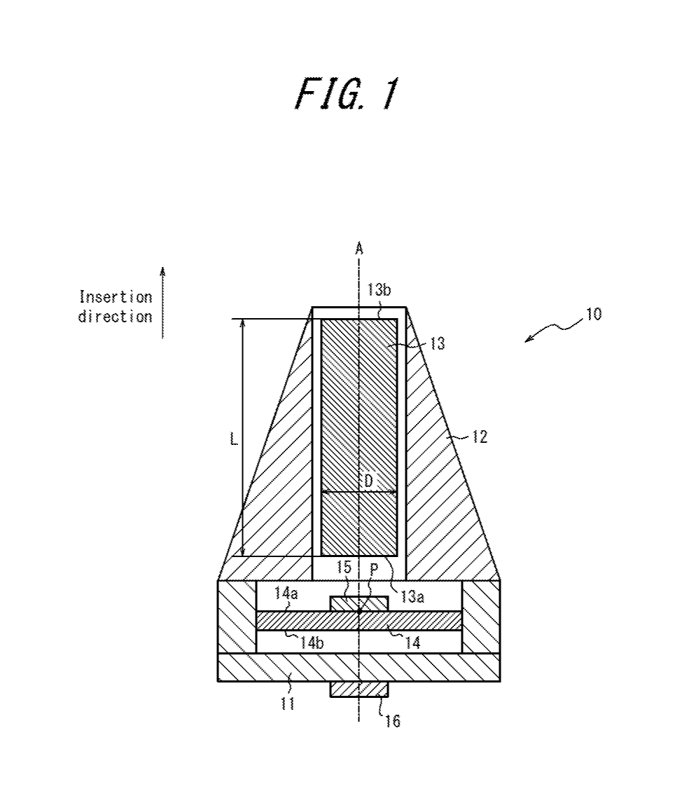

(Embodiment 1) FIG. 1 is a schematic diagram illustrating the internal structure of a measurement apparatus 10 according to Embodiment 1 of the present disclosure. The measurement apparatus 10 is an apparatus to be inserted into a subject's ear canal for use. The outer shape of the measurement apparatus 10 may be configured as a canal type earphone. The measurement apparatus 10 is configured to measure biological information when inserted in the subject's ear. The biological information may be information that can be acquired by a biological sensor described later. Hereinafter, the biological information will be described as the subject's body temperature. The measurement apparatus 10 also functions as an earphone. Thus, in a state in which the measurement apparatus 10 is inserted in the subject's ear (hereinafter, also referred to as “wearing state”), the subject can listen to sound outputted from the measurement apparatus 10 while measuring body temperature with the measurement apparatus 10.

As schematically illustrated in FIG. 1 as an example, the measurement apparatus 10 includes a holder 11, an earpiece 12, a metal tube 13, a substrate 14, a measurement unit 15, and a vibrator 16.

The holder 11 holds and protects various mechanisms included in the measurement apparatus 10. The holder 11 may be made of a hard member such as a resin or the like. When the measurement apparatus 10 is connected to an external device configured to control sound output in a wired manner, a cable for connection with the external device may extend from the holder 11. The holder 11 and the earpiece 12 are coupled to form the housing as the outer shape of the measurement apparatus 10. The holder 11 is formed so as to surround the substrate 14.

The earpiece 12 is configured to be inserted into the subject's ear canal along the insertion direction indicated by the arrow in FIG. 1. That is, the subject can wear the measurement apparatus 10 by inserting the earpiece 12 into an ear canal. The earpiece 12 may be made of a hard member such as resin or the like. In a state in which the earpiece 12 is inserted in the ear canal of the subject, as will be described later, the earpiece 12 transmits vibration to the subject, thereby allowing the subject to hear sound. The earpiece 12 may, for example, be rotationally symmetrical with respect to the virtual axis A illustrated in FIG. 1. The direction of the axis A may be aligned with the insertion direction indicated by the arrow. As viewed from the insertion direction, a cylindrical space is provided in the center of the earpiece 12. The metal tube 13 is held in this space.

The metal tube 13 is a cylindrical metallic tube which is rotationally symmetrical with respect to the axis A. The metal tube 13 functions as a waveguide for electromagnetic radiation radiated from the subject in the wearing state of the measurement apparatus 10. The electromagnetic radiation may, for example, be infrared radiation. Infrared radiation, for example, is electromagnetic radiation with a wavelength in the range of 1 to 20 μm. Hereinafter, the electromagnetic radiation will be described as infrared radiation.

In order to reduce the emissivity of the inner surface of the metal tube 13, gold plating may be applied thereto. The inner surface of the metal tube 13 may be formed from a material with low emissivity to infrared radiation, such as silver. The length L of the metal tube 13 is longer than the diameter D of the metal tube 13 by a predetermined length or more. For example, the length L may be three times the diameter D or more. The metal tube 13 has a first end 13a and a second end 13b. In the measurement apparatus 10, the metal tube 13 is arranged such that the first end 13a is located at the holder 11 side, and the second end 13b is located at the opening side of the measurement apparatus 10. That is, the second end 13b faces the incident direction of infrared radiation in the wearing state of the measurement apparatus 10.

The substrate 14, for example, as illustrated in FIG. 1, is held in the holder 11 and is orthogonal to the axis A. The substrate 14 has a first surface 14a facing the metal tube 13 and a second surface 14b on the other side.

The measurement unit 15 is configured to acquire infrared radiation radiated from the subject, and output a photoelectric conversion signal for the acquired infrared radiation to a control apparatus which is not illustrated in FIG. 1. The control apparatus is configured to measure the subject's body temperature in accordance with the photoelectric conversion signal outputted from the measurement unit 15. The measurement unit 15 is arranged on the first surface 14a, and may be disposed at the position of an intersection point P.

The vibrator 16 is attached to the holder 11 or the earpiece 12. In the example illustrated in FIG. 1, the vibrator 16 is attached to the holder 11. The vibrator 16 is an actuator configured to vibrate based on a sound signal inputted as an electrical signal. The vibration of the vibrator 16 vibrates the housing (the holder 11 and the earpiece 12) to which the vibrator 16 is attached.

The vibrator 16 may, for example, include a piezoelectric element. A piezoelectric element is an element which, when an electrical signal (voltage) is applied, expands or contracts or bends according to the electromechanical coupling coefficient of a constituent material. As the element, for example, those made of ceramic or crystal may be used. The piezoelectric element may be a unimorph, a bimorph, a laminated piezoelectric element, or the like. A laminated piezoelectric element includes a laminated bimorph element in which bimorphs are laminated, for example, by 16 layers or 24 layers. A laminated piezoelectric element may be composed of a laminated structure of, a plurality of dielectric layers, for example, made of PZT (lead zirconate titanate), and electrode layers arranged between the plurality of dielectric layers. A unimorph expands or contracts when an electrical signal (voltage) is applied. A bimorph bends when an electrical signal (voltage) is applied.

In the wearing state, the vibrator 16 vibrates when a sound signal is applied. The vibration of the vibrator 16 causes the holder 11 to vibrate, and the vibration of the holder 11 transmitted to the earpiece 12 causes the earpiece 12 to vibrate. The measurement apparatus 10 is configured to transmit sound to a subject by the vibration of the earpiece 12, namely, by bone conduction sound. The subject can recognize sound reproduced by the bone conduction sound.

For example, in the measurement apparatus 10 having the structure illustrated in FIG. 1, when a speaker is arranged inside the holder 11 for outputting sound, sound outputted from the speaker is configured to pass through the inside of the metal tube 13 and then be outputted to the outside of the measurement apparatus 10. Here, as described above, in the present embodiment, the length L of the metal tube 13 is longer than the diameter D thereof by a predetermined amount or more. The metal tube 13 has acoustic characteristics such that amplitudes at predetermined frequencies (resonance frequencies) are amplified by resonance.

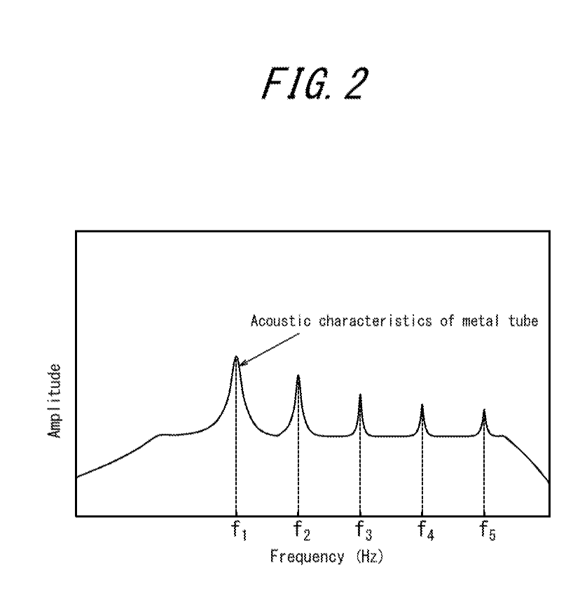

For example, as indicated by the solid line in FIG. 2, the metal tube 13 has acoustic characteristics such that amplitudes at the predetermined frequencies f_{1,} f_{2,} f_{3,} f₄ and f₅ are higher than those in other frequency ranges. Thus, when the characteristics of the sound inputted to the first end 13a of the metal tube 13 are uniform, sound at the predetermined frequencies f₁, f_{2,} f_{3,} f₄ and f₅ is amplified more than sound in other frequency ranges due to the acoustic characteristics of the metal tube 13, and is then outputted from the second end 13b. That is, in this case, sound outputted at the predetermined frequencies f_{1,} f_{2,} f_{3,} f₄ and f₅ is be stronger than that outputted in other frequency bands. From the perspective of the subject listening to the source, it is undesirable for the sound pressure per frequency to differ greatly.

On the other hand, as described above, in the measurement apparatus 10 according to the present embodiment, sound is transmitted to the subject by bone conduction sound. That is, the measurement apparatus 10 does not output sound through the metal tube 13. Thus, the measurement apparatus 10 makes it easier to output good sound to a subject compared to the case where sound is outputted through the metal tube 13.

Next, measurement of body temperature by the measurement apparatus 10 will be described in detail. The measurement apparatus 10 is configured to acquire infrared radiation radiated from a subject by the measurement unit 15. Infrared radiation radiated from a subject is configured to enter the metal tube 13 from the second end 13b and then be received by the measurement unit 15. In the present embodiment, the length L of the metal tube 13 is longer than the diameter D thereof by a predetermined amount or more; and the measurement unit 15 is arranged at the inner side of the metal tube, namely, the 13a side.

It is assumed that the measurement unit 15 is able to receive infrared radiation radiated from the wall surface of an ear canal, when the length L of the metal tube 13 is not longer than the diameter D thereof by the predetermined amount, or when the measurement unit 15 is arranged at the second end 13b side of the metal tube 13. The wall surface of an ear canal, however, is easily affected by outside air. Thus, infrared radiation received by the measurement unit 15 in this case is not necessarily based on the actual body temperature of the subject, but may be based on a body temperature affected by outside air temperature. Consequently, it would be difficult to accurately measure a subject's body temperature.

On the contrary, according to the measurement apparatus 10 of the present embodiment, the directivity of infrared radiation to be received by the measurement unit 15 can be increased, compared to the case that the length L of the metal tube 13 is not longer than the diameter D thereof by the predetermined amount, or the case that the measurement unit 15 is arranged at the second end 13b side of the metal tube 13. This allows the measurement unit 15 to receive infrared radiation at a specific portion of the ear of the subject when wearing the measurement apparatus 10.

More particularly, for example, the measurement unit 15 can receive infrared radiation radiated from the subject's eardrum. The eardrum, by virtue of being located in an inner part of the ear, is not as affected by outside air. Thus, by receiving infrared radiation radiated from the eardrum, the measurement apparatus 10 can easily and accurately measure a subject's body temperature accurately.

In this manner, the measurement apparatus 10 according to the present embodiment makes it easier to output good sound while improving the accuracy of body temperature detection.

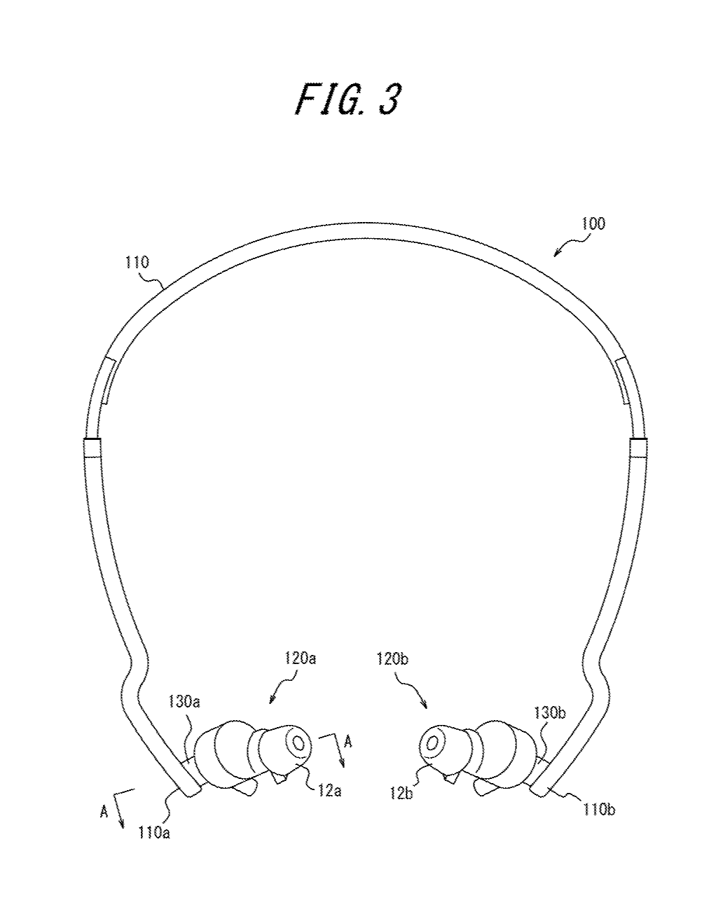

FIG. 3 is a schematic diagram illustrating the appearance of a measurement instrument 100 provided with the measurement apparatus 10. The measurement instrument 100, as an example illustrated in FIG. 3, can be configured as a headband type earphone. The measurement instrument 100 includes a wearing portion 110, a first measurement portion 120a and a second measurement portion 120b, and a first connector 130a and a second connector 130b. The measurement instrument 100 may, for example, be wearable on a subject's head.

The wearing portion 110 is a mechanism for keeping the wearing state of the measurement instrument 100 to a subject. In the present embodiment, the wearing portion 110, for example, has an arch shape as illustrated in FIG. 3. A subject can wear the measurement instrument 100 by sandwiching the head with the wearing portion 110. The wearing portion 110 may, for example, have a mechanism such that the length of the wearing portion 110 can be adjusted according to the size of the subject's head. The wearing portion 110 may be formed from plastic or the like.

The first measurement portion 120a is provided at the first end 110a of the wearing portion 110, and is connected to the wearing portion 110 via the first connector 130a; and the second measurement portion 120b is provided at the second end 110b of the wearing portion 110, and is connected to the wearing portion 110 via the second connector 130b. That is, the wearing portion 110, the first measurement portion 120a and the second measurement portion 120b, and the first connector 130a and the second connector 130b, are configured as one measurement instrument 100 which is connected as a whole. The first connector 130a is formed at a portion connecting the first measurement portion 120a and the first end 110a of the wearing portion 110, and the second connector 130b is formed at a portion connecting the second measurement portion 120b and the second end 110b of the wearing portion 110.

The first measurement portion 120a and the second measurement portion 120b are the measurement apparatus 10 illustrated in FIG. 1 realized in the measurement instrument 100. That is, the first measurement portion 120a and the second measurement portion 120b have a configuration similar to that of the measurement apparatus 10. The first measurement portion 120a and the second measurement portion 120b may, for example, be configured symmetrically and have similar functions. In the present description, when there is no need to distinguish the first measurement portion 120a and the second measurement portion 120b, these are collectively referred to as measurement portion 120.

The first measurement portion 120a is configured to measure body temperature in the right ear when the earpiece 12a is inserted in the concha of the right ear; and the second measurement 120b is configured to measure body temperature in the left ear when the earpiece 12b is inserted in the concha of the left ear.

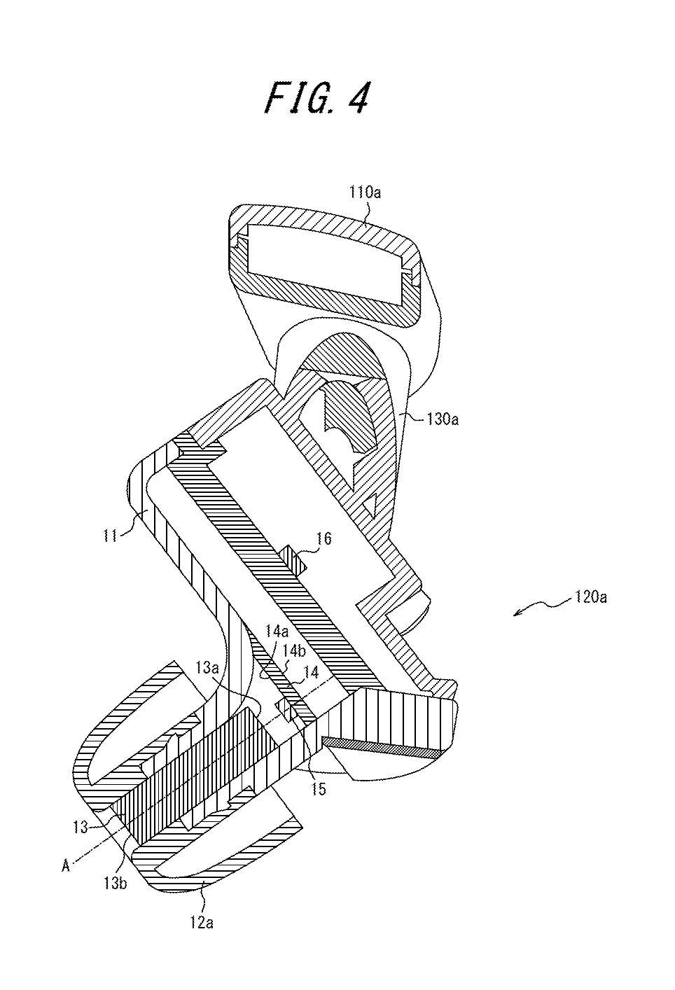

FIG. 4 is a cross-sectional view taken from line A-A of the measurement instrument 100 illustrated in FIG. 3. As illustrated in FIG. 4, the outer shape of the measurement portion 120a is formed by coupling the holder 11 and the earpiece 12a. As viewed from the insertion direction, a cylindrical space is provided in the center of the earpiece 12a. In this space, the metal tube 13 is held. The substrate 14 is provided inside the holder 11. The measurement unit 15 is arranged on the first surface 14a of the substrate 14 at a position which intersects the axis A. The vibrator 16 is arranged inside the holder 11. Since the structure of the second measurement portion 120b may be configured symmetrically with the first measurement portion 120a, the description thereof will be omitted.

With the above configuration, it is easy for the measurement instrument 100 to accurately measure the body temperature of a subject wearing the measurement instrument 100 and output good sound.

It has been described that the measurement instrument 100 illustrated in FIG. 3 includes two measurement portions, namely, the first measurement portion 120a and the second measurement portion 120b. However, the measurement instrument 100 may, for example, include only one measurement portion. For example, one of the first measurement portion 120a and the second measurement portion 120b has the functions of measuring subject's body temperature and outputting sound, and the other one only has the function of outputting sound. In addition, the measurement instrument 100 may determine the subject's body temperature based on body temperature values acquired from two measurement portions, namely, the first measurement portion 120a and the second measurement portion 120b, for example, by calculating the average value of two body temperature values.

The measurement instrument 100 illustrated in FIG. 3 may be configured such that one of the first measurement portion 120a and the second measurement portion 120b is the measurement apparatus 10 according to the present embodiment, and the other one is a measurement apparatus for measuring biological information other than body temperature. For example, the other one may be configured as a blood flow measurement apparatus to be inserted in a subject's ear to measure the blood flow of the subject. The blood flow measurement apparatus may, for example, be configured to irradiate laser light to the subject and calculate the blood flow based on the reflected light of the laser light. In another example, the other one may be configured as an oxygen saturation measurement apparatus to be inserted in a subject's ear to measure the oxygen saturation of the subject. The oxygen saturation measurement apparatus may, for example, be configured to irradiate visible light and infrared light to the subject and calculate the oxygen saturation based on the ratio of the intensities of the visible light and the infrared light passing through the subject or reflected from the subject.

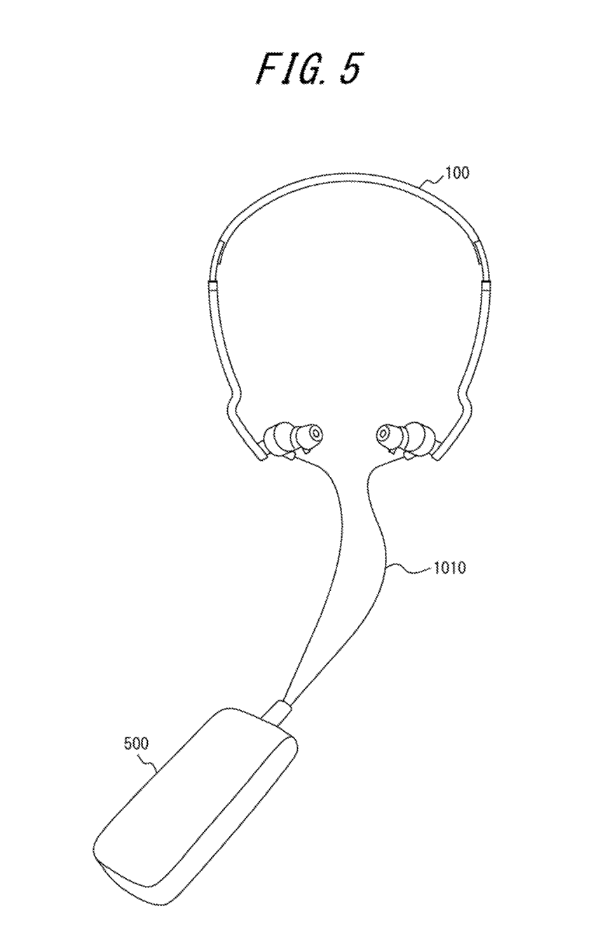

The measurement apparatus 10 can be mounted on a measurement instrument. FIG. 5 is a schematic diagram illustrating a connection configuration of the measurement instrument 100 and a control apparatus 500. As illustrated in FIG. 5, the control apparatus 500 is connected to the measurement instrument 100 by a cord 1010. The control apparatus 500 and the measurement instrument 100 transmit and receive information and power via the cord 1010. In addition to the wired cord 1010, the control apparatus 500 and the measurement instrument 100 may transmit and receive information and power in a wireless manner. This wireless manner can be anything such as Bluetooth® (Bluetooth is a registered trademark in Japan, other countries, or both), infrared radiation, NFC (Near Field Radio Communication), or the like. Further, the measurement instrument 100 may include a battery such as a cell or the like.

The measurement instrument 100 can be configured as, for example, a headband type earphone. The measurement instrument 100 may be wearable, for example, on a subject's head. Further, the measurement instrument 100 may include the measurement apparatus 10 in a part to be inserted into an ear.

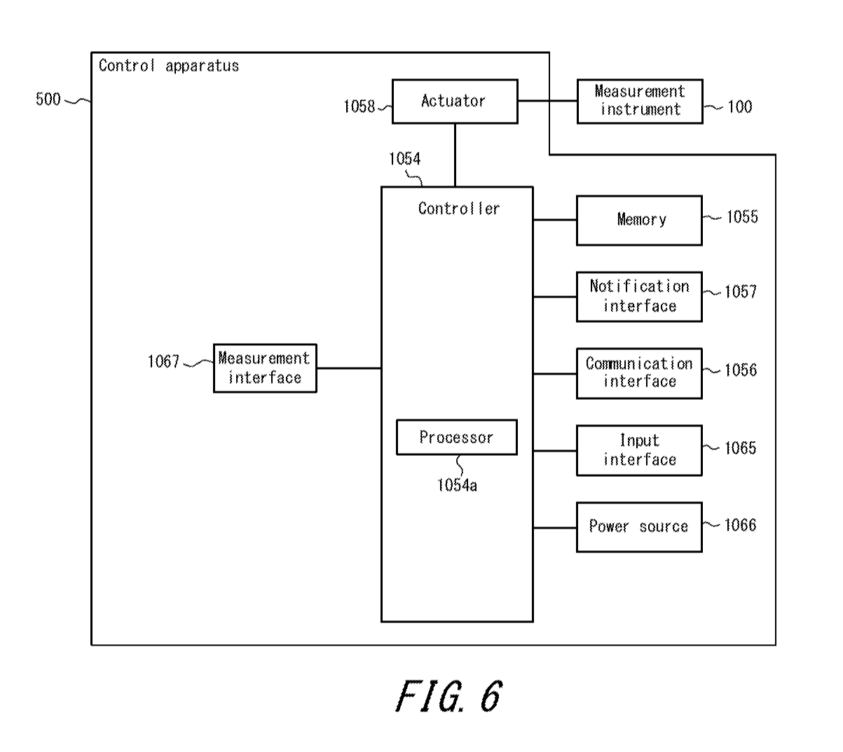

Next, the internal structure of the control apparatus 500 illustrated in FIG. 5 will be described with reference to FIG. 6. FIG. 6 illustrates an example of functional blocks included in the control apparatus 500 illustrated in FIG. 5. The control apparatus 500 is configured to control the sound output of the measurement apparatus 10.

The control apparatus 500 includes a measurement interface 1067, a controller 1054, a memory 1055, a communication interface 1056, a notification interface 1057, an actuator 1058, an input interface 1065, and a power source 1066 as functional units.

The controller 1054 includes at least one processor 1054a for controlling and managing the whole control apparatus 500, including each functional block of the control apparatus 500. The controller 1054 includes at least one processor 1054a such as a CPU (Central Processing Unit) configured to execute a program prescribing a control procedure, and thus enables its functions. Such a program is stored in, for example, the memory 1055 or an external storage medium connected to the control apparatus 500.

According to various embodiments of the present disclosure, the at least one processor 1054a may be performed as a single integrated circuit (IC), or a plurality of communicably connected integrated circuits and/or discrete circuits. Further, the at least one processor 1054a can be performed in accordance with various known techniques.

In one embodiment according to the present disclosure, the processor 1054a may include one or more circuits or units configured to perform one or more data calculation procedures or processes by executing instructions stored in an associated memory. In another embodiment according to the present disclosure, the processor 1054a may be firmware, for example, a discrete logic component, configured to perform one or more data calculation procedures or processes.

According to various embodiments of the present disclosure, the processor 1054a may include one or more processors, a controller, a microprocessor, a microcontroller, an application specific integrated circuit (ASIC), a digital signal processor, a programmable logic device, a field programmable gate array, any combinations of these devices or configurations, or any combinations of other known devices or configurations; and thus perform the functions as the controller 1054 described later.

The controller 1054 controls the output of sound to be transmitted to a subject by controlling the vibration of the vibrator 16 of the measurement apparatus 10. Further, the controller 1054 measures or calculates the subject's body temperature based on biological information measured by the measurement unit 15 of the measurement apparatus 10.

The memory 1055 can be constituted by a semiconductor memory, a magnetic memory, or the like. The memory 1055 stores various kinds of information and/or a program for operating the control apparatus 500 and the like. The memory 1055 may also function as a working memory. The memory 1055 may, for example, store a body temperature calculated by the controller 1054.

In the memory 1055, music information and other sound information to be transmitted to a subject by vibration of the vibrator 16 of the measurement instrument 100 is stored. The controller 1054 is configured to extract predetermined sound information from the memory 1055 based on an input operation or the like by the user, and cause the vibrator 16 of the measurement instrument 100 to vibrate via the actuator 1058. Then, the vibrator 16 vibrates based on the received sound information to transmit sound to the subject.

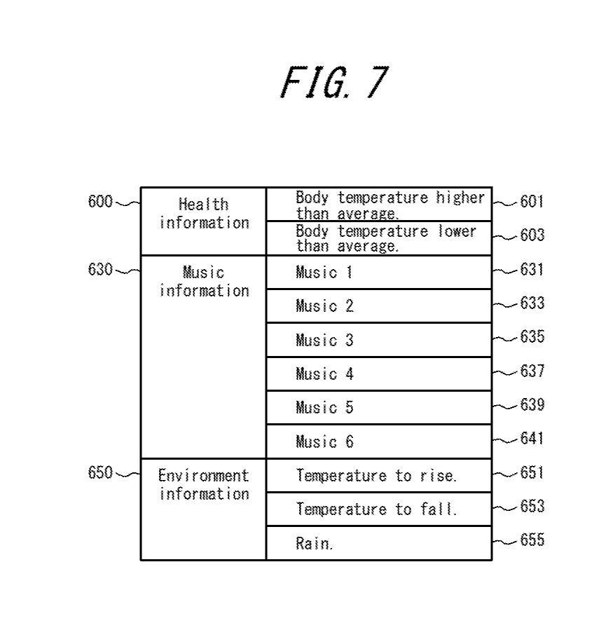

Here, sound data to be stored in the memory 1055 will be described with reference to FIG. 7. FIG. 7 is a conceptual diagram of sound data to be stored in the memory 1055.

As illustrated in FIG. 7, sound data of health information 600, music information 630 and environment information 650 are stored in the memory 1055. The health information 600 includes, for example, information 601 for notification of “the body temperature is higher than average”, and information 603 for notification of “the body temperature is lower than average”. The average here may, for example, be an average of a typical adult or an average of the subject. Further, in the present disclosure, the health information 600 is not limited to those illustrated in FIG. 7, and may be any other sound information.

The music information 630 includes, for example, pieces of music information 631, 633, 635, 637, 639, and 641. In the present disclosure, the music information is not limited to six pieces, and may be any number.

The environment information 650 includes, for example, sound information related to the surrounding environment of the measurement instrument 100, such as information 651 for notification of a temperature rise after a predetermined time, information 653 for notification of a temperature fall after a predetermined time, information 655 for notification of a rainfall after a predetermined time, and the like. In the present disclosure, the environment information 650 is not limited to those illustrated in FIG. 7, and may be any other environment information, such as information for notification of a snowfall or a fog, information related to road conditions such as a traffic jam or a traffic accident, and information related to operation status of a train, a bus, an airplane, and the like.

The communication interface 1056 transmits and receives various kinds of information by communicating with an external device via a network. The external device that the communication interface 1056 communicates with may, for example, be a server, a PC (Personal Computer), a mobile phone, a smartphone, a tablet, a wristwatch, a massage machine, any other devices, or any combinations of these. The communication interface 1056 transmits and receives information to and from the external device using a network configured in a wireless manner, a wired manner, or a combination of these. For example, the communication interface 1056 can use Bluetooth, infrared radiation, NFC, wireless LAN (Local Area Network), wired LAN, any other communication mediums, or any combinations of these. The communication interface 1056 acquires biological information related to body temperature measured by the measurement unit 15 by communicating with the measurement instrument 100.

The notification interface 1057 notifies information by sound, light, image, vibration, or other appropriate methods. The notification interface 1057 may be constituted by, for example, a liquid crystal display, a speaker, a LED (Light Emitting Diode), a vibrator, any other notification members, or any combinations of these. The notification interface 1057 may notify, for example, information related to a measured body temperature.

The actuator 1058 is configured to output a signal for vibrating the vibrator 16 based on a control signal from the controller 1054.

The input interface 1065 is configured to receive input operations made by the subject using the control apparatus, and is constituted by, for example, operation buttons (operation keys). The input interface 1065 may also be constituted by a touch panel, and on a part of the display device, operation keys may be displayed to receive touch operation input by a subject.

The power source 1066 is a battery for supplying power for the control apparatus 500 to perform various operations.

The measurement interface 1067 is configured to measure the surrounding environment and the like. In the present disclosure, the measurement interface 1067 is a thermometer for measuring temperature at a predetermined portion. In the present disclosure, since the predetermined portion is a part of human body, thus, the temperature is body temperature, and the thermometer is a clinical thermometer. The measurement interface 1067 may, for example, be a clinical thermometer, a thermometer, a hygrometer, an altimeter, any other measurement instruments, or any combinations of these. The measurement interface 1067 is capable of measuring body temperature, temperature, humidity, altitude and other data in an environment in which the control apparatus 500 is arranged.

The controller 1054 may acquire weather information for a date and time when a body temperature is measured from a weather information server, which is connected to the control apparatus 500 by a network, via the notification interface 1056, and then transmit the acquired information to a server 1000.

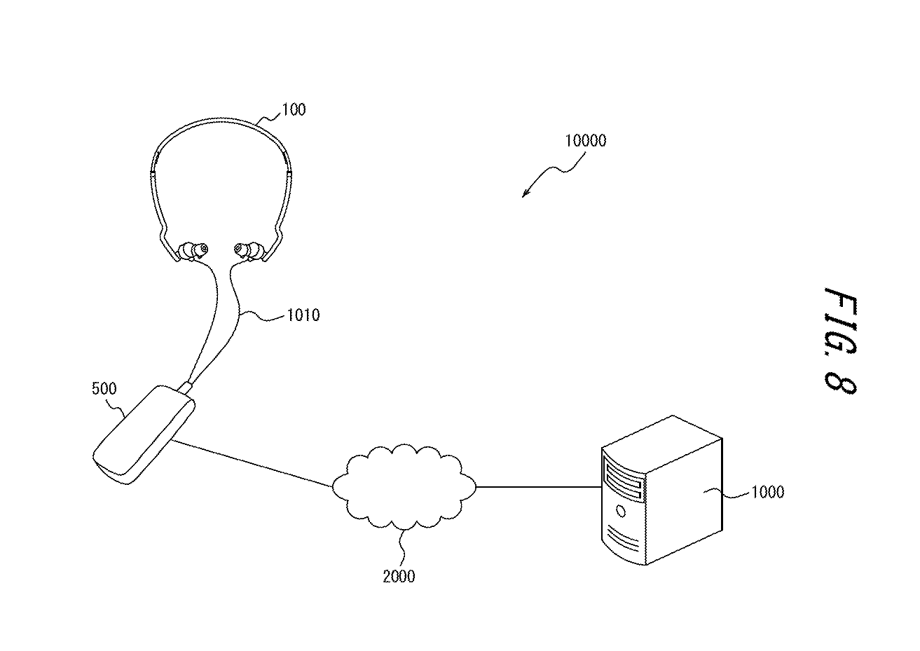

Next, a measurement system 10000 in which the measurement instrument 100 illustrated in FIG. 5 is used will be described with reference to FIG. 8. As illustrated in FIG. 8, the control apparatus 500 connected to the measurement instrument 100 is also connected to the server 1000 via a network 2000. In the measurement system 10000, the server 1000 may be a PC, a mobile phone, a smartphone, a tablet, a wristwatch, a massage machine, any other devices, or any combination of these.

In the present disclosure, the measurement instrument 100 transmits and receives information to and from the server 1000 connected to the network 2000 via the control apparatus 500. However, the measurement instrument 100 may be directly connected to the network 2000 to transmit and receive information to and from the server 1000.

The network 2000 in the present disclosure may be the Internet. However, the network 2000 is not limited to this, and may be any appropriate network. Further, the network 2000 may be configured in a wireless manner, a wired manner, or a combination of these. That is, the control apparatus 500 and the server 1000 may be connected by the network 2000 configured in a wired manner, a wireless manner, or a combination of these, to transmit and receive information to and from each other.

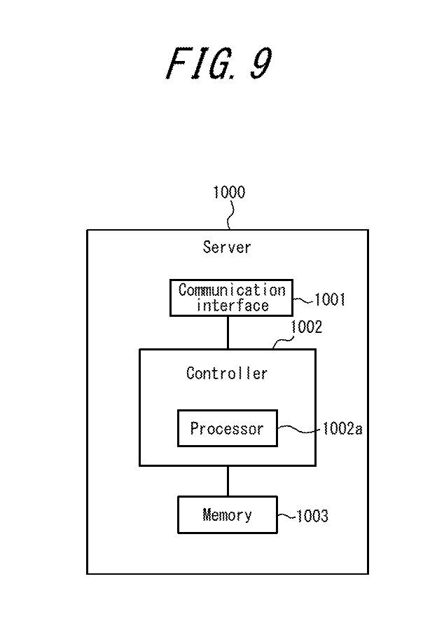

Next, the internal structure of the server 1000 illustrated in FIG. 8 will be described with reference to FIG. 9. FIG. 9 is a functional block diagram illustrating the schematic configuration of the server 1000 in FIG. 8. The server 1000 includes a communication interface 1001, a controller 1002, and a memory 1003.

The communication interface 1001 transmits and receives various kinds of information by communicating with the control apparatus 500 via the network 2000. The network 2000 may be configured in a wireless manner, a wired manner, or a combination of these. For example, the communication interface 1001 can use Bluetooth, infrared radiation, NFC, wireless LAN, wired LAN, WAN (Wide Area Network), the Internet, any other communication mediums, or any combination of these to perform communication. In the present embodiment, the communication interface 1001 uses the Internet to communicate with the control apparatus 500.

The controller 1002 includes at least one processor 1002a for controlling and managing the whole server 1000, including each functional block of the server 1000. The controller 1002 includes at least one processor 1002a such as a CPU configured to execute a program prescribing a control procedure, and thus enables its functions. Such a program is stored in, for example, the memory 1003 or an external storage medium connected to the server 1000.

According to various embodiments of the present disclosure, the at least one processor 1002a may be performed as a single integrated circuit (IC), or as a plurality of communicably connected integrated circuits and/or discrete circuits. Further, the at least one processor 1002a can be implemented in accordance with various known techniques.

In one embodiment according to the present disclosure, the processor 1002a may include one or more circuits or units configured to perform one or more data calculation procedures or processes by executing instructions stored in an associated memory. In another embodiment according to the present disclosure, the processor 1002a may be firmware, for example, a discrete logic component, configured to perform one or more data calculation procedures or processes.

According to various embodiments of the present disclosure, the processor 1002a may include one or more processors, a controller, a microprocessor, a microcontroller, an application specific integrated circuit (ASIC), a digital signal processor, a programmable logic device, a field programmable gate array, any combination of these devices or configurations, or any combination of other known devices or configurations; and thus perform the functions as the controller 1002.

The memory 1003 can be constituted by a semiconductor memory, a magnetic memory, or the like. The memory 1003 stores various kinds of information and/or a program for operating the server 1000 and the like. The memory 1003 may also function as a working memory. The memory 1003 may store, for example, various kinds of information acquired by the server 1000 from the communication interface 1001.

The server 1000 is configured to receive information related to body temperature from the control apparatus 500, and analyze the received information. Then, the server 1000 transmits a result indicating the analysis to the control apparatus 500. Further, the server 1000 can receive other information such as air temperature, weather, disaster information and the like via the network 2000.

The server 1000 may store the information received from the control apparatus 500 in the memory 1003, as standalone information or in association with user's personal information. The server 1000 may be managed by a company to which the user belongs, a government, a public organization, a medical institution, a health management organization, an insurance company, or the like.

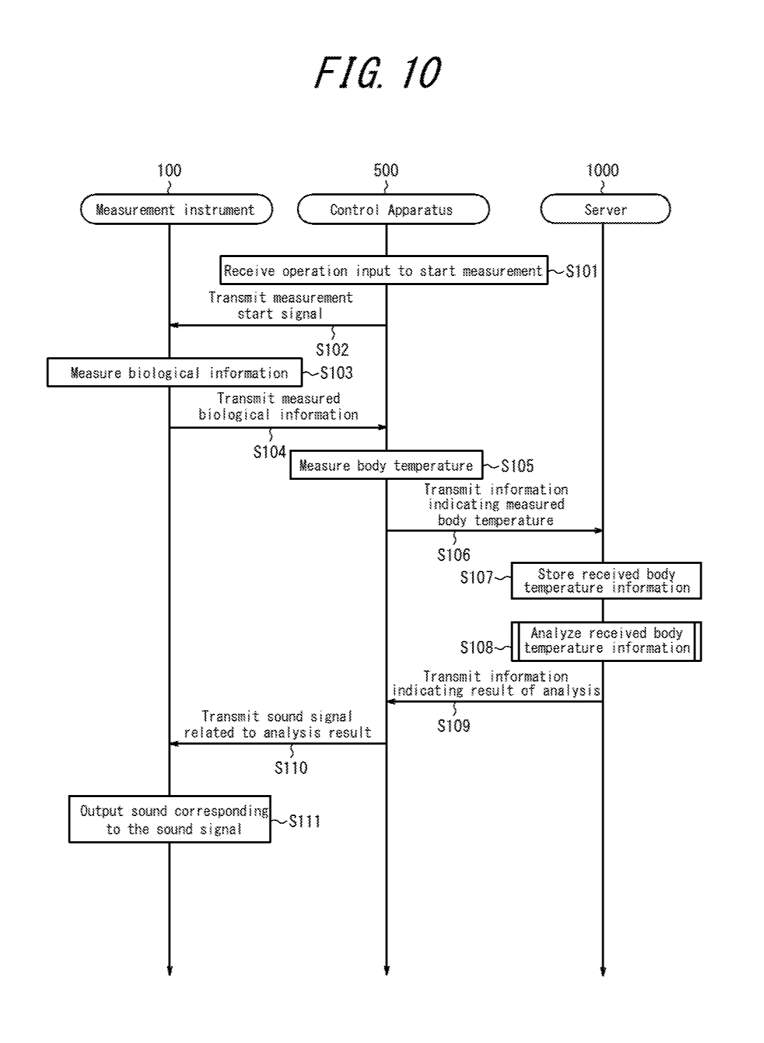

In the example illustrated in FIG. 8, there is one server 1000. However, the present disclosure in not limited to this, and the server 1000 may be any number greater than or equal to one. In this case, a plurality of servers 1000 may be configured to perform the same processing, or perform the processing illustrated in FIG. 10 described later in a distributed manner, or perform a combination of these.

Next, an example of processing by the measurement system 10000 will be described. FIG. 10 is a sequence chart illustrating an example of processing by the measurement system 10000. The sequence illustrated in FIG. 10 may be started, for example, according to a state in which a user is wearing the measurement instrument 100.

Initially, the control apparatus 500 receives an operation input indicating measurement start from a subject (Step S101).

Upon receiving the operation input, the control apparatus 500 transmits a measurement start signal to the measurement instrument 100 (Step S102).

Based on the measurement start signal, the measurement instrument 100 measures biological information using the measurement unit 15 of the measurement apparatus 10 (Step S103).

Then, the measurement instrument 100 transmits the biological information measured by the measurement unit 15 to the control apparatus 500 (Step S104).

Based on the biological information acquired from the measurement instrument 100, the control apparatus 500 measures the subject's body temperature (Step S105).

Then, the control apparatus 500 transmits information related to the measured body temperature (hereinafter, also referred to as “body temperature information”) to the server 1000 (Step S106).

Upon receiving the body temperature information from the control apparatus 500, the server 1000 stores the received body temperature information in the memory 1003 (Step S107).

Further, the server 1000 analyzes the body temperature information received from the control apparatus 500 (Step S108). Details of the body temperature information analysis will be described later.

Then, the server 1000 transmits information indicating a result of the analysis to the control apparatus 500 (Step S109).

Based on the received analysis result, the control apparatus 500 transmits a sound signal related to the analysis result to the measurement instrument 100 (Step S110).

Then, the measurement instrument 100 causes the vibrator 16 to vibrate in accordance with the sound signal to transmit sound to the subject, thereby outputting a sound corresponding to the sound signal (Step S111). Through the output of the sound, the subject can be notified of the analysis result.

In Step S110, the control apparatus 500 may output a notification corresponding to the analysis result from the notification interface 1057 without transmitting the sound signal.

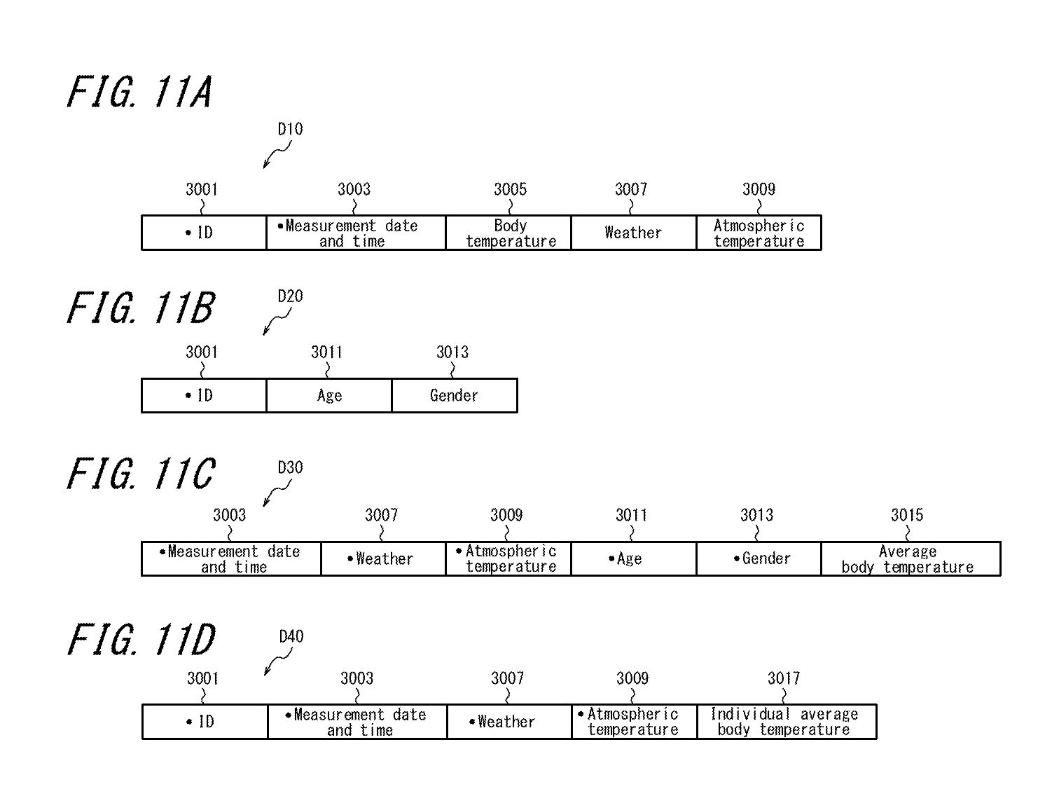

Next, the data to be stored in the server 1000 illustrated in FIG. 8 will be described with reference to FIGS. 11A, 11B, 11C, and 11D. FIGS. 11A, 11B, 11C, and 11D are configuration diagrams illustrating examples of data to be stored in the server 1000 illustrated in FIG. 8. The information illustrated in FIGS. 11A, 11B, 11C, and 11D may be acquired by measurement, by the server 1000 via the network 2000, or by user's input.

FIG. 11A illustrates a data structure D10 with user ID 3001 and measurement date and time 3003 as primary keys. Here, in FIGS. 11A, 11B, 11C, and 11D, a black dot is attached to each primary key. At least one or more data of values corresponding to attributes of each column are stored in each column.

User ID 3001 is a value that uniquely corresponds to a user. Measurement date and time 3003 is a value of the date and time when the biological information was measured. The measurement date and time may have a certain range, such as an hour (for example, from 0:00 to 1:00), a time period (for example, in the morning), a date (for example, January 1), a month (for example, January), a plurality of months (for example, from January to March), a year (for example, 2100), or the like.

Body temperature 3005 is information related to a body temperature measured at the measurement date and time 3001 by a user identified by the user ID 3001. Weather 3007 is a value of the weather at the measurement date and time 3003. The temperature 3009 is information indicating the atmospheric temperature at measurement date and time 3003.

FIG. 11B illustrates a data structure D20 with the user ID3001 as a primary key. Age 3011 is the age of the user identified by user ID 3001. Gender 3013 is the gender of the user identified by user ID 3001.

FIG. 11C illustrates a data structure D30 with measurement date and time 3003, weather 3007, temperature 3009, age 3011, and gender 3013 as primary keys. Average body temperature 3015 is an average value of body temperatures measured in a user group identified by measurement date and time 3003, weather 3007, temperature 3009, age 3011, and gender 3013. That is, the average body temperature 3015 is an average body temperature of a group to which the user belongs.

FIG. 11D illustrates a data structure D40 with user ID 3001, measurement date and time 3003, weather 3007, and temperature 3009 as primary keys. Individual average body temperature 3017 is an average value of body temperatures of a specific user measured in a predetermined situation, and is identified by user ID 3001, measurement date and time 3003, weather 3007, and temperature 3009.

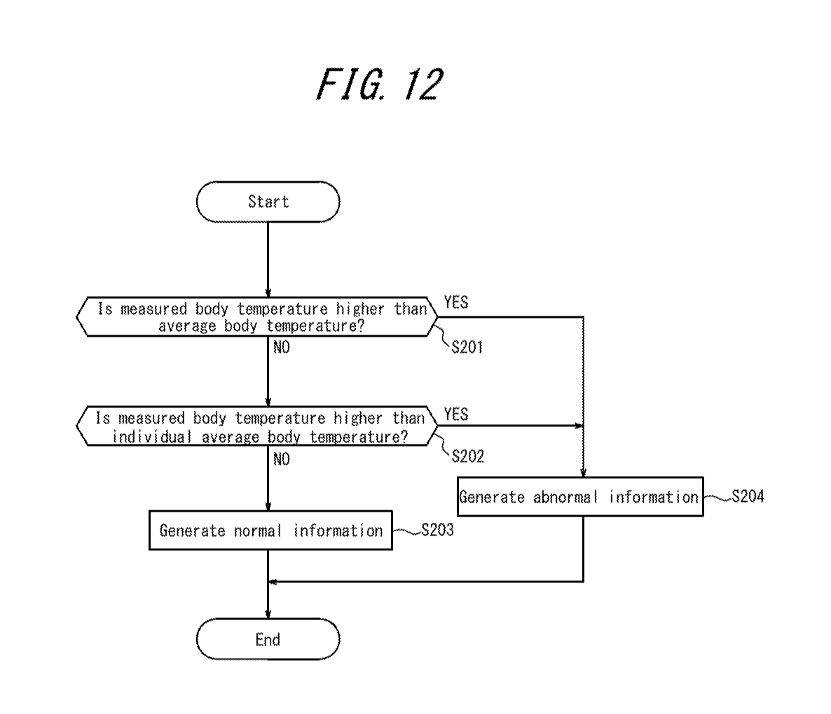

Next, analysis processing of body temperature information by the server 1000 illustrated in FIG. 8 will be described in detail with reference to FIG. 12. FIG. 12 is a flowchart illustrating an example of analysis processing of biological information by the server 1000 illustrated in FIG. 8. That is, FIG. 12 is a flowchart illustrating an example of the detailed processing of Step S108 illustrated in FIG. 10. The processing illustrated in FIG. 12 can be realized, for example, by the processor 1002a illustrated in FIG. 9 cooperating with the program stored in the memory 1003.

As described above, the server 1000 receives the body temperature information transmitted from the control apparatus 500 via the network 2000 in Step S106 illustrated in FIG. 10. At this time, the server 1000 may further acquire information regarding user ID, measurement date and time, weather, and temperature from the control apparatus 500.

Based on the acquired information, in Step S107, the server 1000 generates the data structure D10 illustrated in FIG. 11A and stores the same in the memory.

Then, the server 1000 determines whether the subject's body temperature indicated by the received body temperature information (hereinafter, also referred to as “measured body temperature”) is higher than the average body temperature in the data structure D30 illustrated in FIG. 11C with reference to the data structure D10, the data structure D20, and the data structure D30 (Step S201).

When the measured body temperature is lower than the average body temperature in the data structure D30 illustrated in FIG. 11C (Step S201: No), the server 1000 then determines whether the measured body temperature is higher than the individual average body temperature in the data structure D40 illustrated in FIG. 11D with reference to the data structure D10 and the data structure D40 (Step S202).

When the measurement body temperature is lower than the individual average body temperature in the data structure D40 illustrated in FIG. 11D (Step S202: No), the server 1000 then generates normal information indicating that the measured result is normal (Step S203). This is because when the measured body temperature is lower than the individual average body temperature, it can be determined to be normal. The generated normal information is then transmitted to the control apparatus 500 as a result of the analysis in Step S109 illustrated in FIG. 10.

On the other hand, when the measured temperature is higher than the individual average body temperature in the data structure D40 illustrated in FIG. 11D (Step S202: Yes), the server 1000 generates abnormal information indicating that the measured result is abnormal (Step S204). This is because when the measured temperature is higher than the individual average body temperature, it can be determined to be abnormal. The generated abnormal information is then transmitted to the control apparatus 500 a result of the analysis in Step S109 illustrated in FIG. 10.

Further, when the measured body temperature is higher than the average body temperature in the data structure D30 illustrated in FIG. 11C (Step S201: Yes), the server 1000 generates abnormal information indicating that the measured result is abnormal (Step S204). This is because when the measured body temperature is higher than the average body temperature, it can be determined to be abnormal. The generated abnormal information is then transmitted to the control apparatus 500 a result of the analysis in Step S109 illustrated in FIG. 10.

Upon receiving the analysis information from the server 1000, the control apparatus 500 transmits a sound signal related to the analysis result, based on the normal information or the abnormal information included in the analysis information, to the measurement instrument 100 (Step S110). Then, the measurement instrument 100 outputs a sound corresponding to the sound signal by vibration of the vibrator 16 (Step S111). In addition to sound, the measurement instrument 100 may notify the analysis result by light, image, vibration, other notification means, or any combination of these.

(Embodiment 2) Next, Embodiment 2 will be described. In Embodiment 2, a smartphone 3000 is used instead of the control apparatus 500, which is used in the measurement system of Embodiment 1. In Embodiment 2, the same reference numbers are assigned to members similar to those of Embodiment 1, and the description thereof will be omitted.

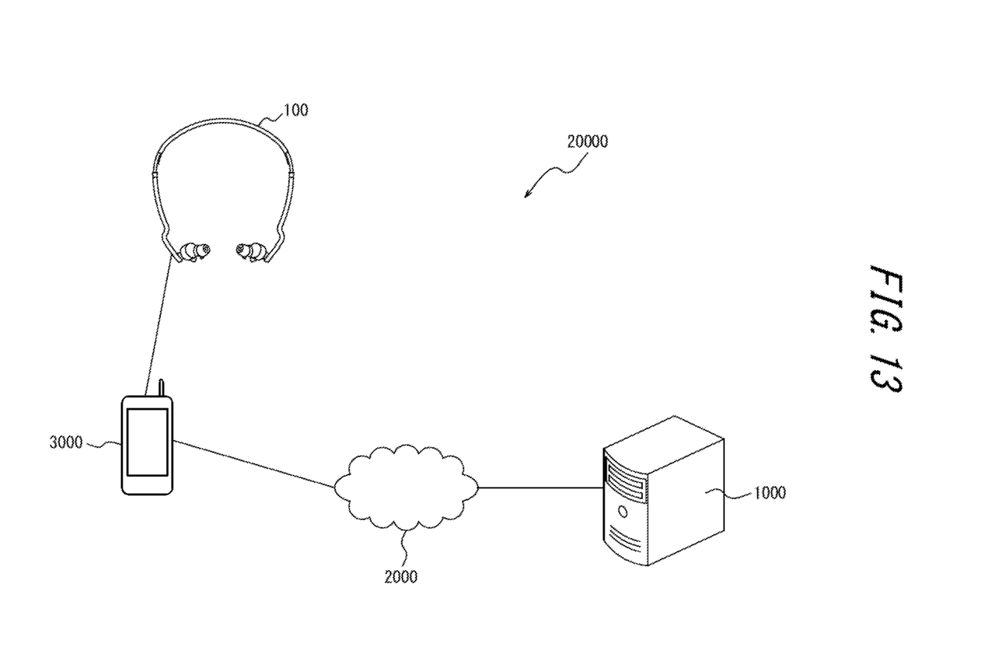

FIG. 13 illustrates a configuration example of a measurement system 20000 according to Embodiment 2. As illustrated in FIG. 13, compared to the measurement system 10000 according to Embodiment 1, the smartphone 3000 instead of the control apparatus 500 is used in the measurement system 20000 according to the present embodiment. The smartphone 3000 is communicably connected to the measurement instrument 100 in a wireless manner, a wired manner, or a combination of these. In addition, the smartphone 3000 is communicably connected to the server 1000 via the network 2000.

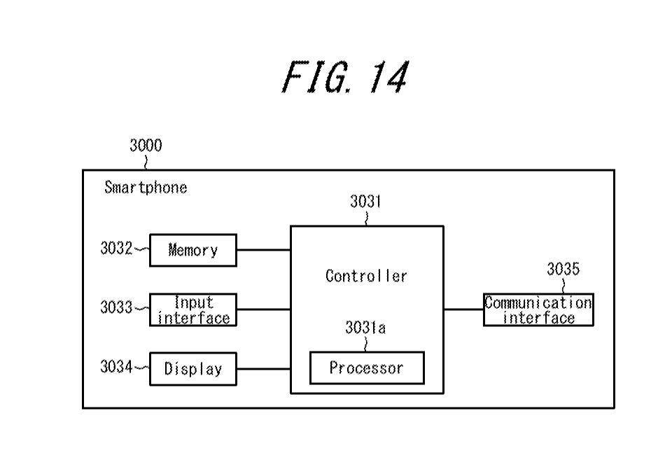

Next, an example of the functional blocks included in the smartphone 3000 illustrated in FIG. 13 will be described with reference to FIG. 14. The smartphone 3000 includes a controller 3031, a memory 3032, an input interface 3033, a display 3034, and a communication interface 3035 as function units.

The controller 3031 includes at least one processor 3031a for controlling and managing the whole smartphone 3000, including each functional block of the smartphone 3000. The controller 3031 includes at least one processor 3031a such as a CPU configured to execute a program prescribing a control procedure, and thus enables its functions. Such a program is stored in, for example, the memory 3032 or an external storage medium connected to the smartphone 3000. As the specific configuration of the processor 3031a, those enumerated in the description of the processor 1054a can be used.

The controller 3031 may perform the same processing with the controller 1054 of the control apparatus 500 of Embodiment 1.

The memory 3032 can be constituted by a semiconductor memory, a magnetic memory, or the like. The memory 3032 stores various kinds of information, a program for operating the smartphone 3000, and the like. The memory 3032 may also function as a working memory.

The input interface 3033 is configured to receive operation input by a user of the smartphone 3000 (for example, a subject), and is constituted by, for example, operation buttons (operation keys). The input interface 3033 may also be constituted by a touchscreen, and on a part of the display device which is the display 3034, an input area may be displayed to receive touch operation input by a user.

The display 3034 is constituted by a well-known display, for example, a LCD (Liquid Crystal Display), an OELD (Organic Electro-Luminescence Display), or an IELD (Inorganic Electro-Luminescence Display).

The communication interface 3035 transmits and receives various kinds of information by communicating with the measurement instrument 100 and the server 1000. The communication interface 3035 can transmit and receive information using a network configured in a wireless manner, a wired manner, or a combination of these.

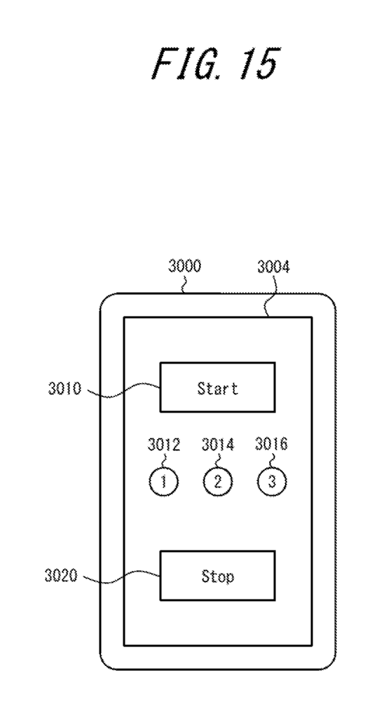

Next, an operation screen of the smartphone illustrated in FIG. 13 will be described with reference to FIG. 15. FIG. 15 is a schematic diagram of the operation screen of the smartphone 3000 illustrated in FIG. 13.

As illustrated in FIG. 15, a start button 3010, various functional buttons 3012, 3014, and 3016, and a stop button 3020 are displayed on the display 3034 of the smartphone 3000. The display 3034 of the smartphone 3000 is a touch panel display. When a user touches an image displayed on the display 3034 with a finger or the like, an operation input corresponding to the image is performed. For example, a subject can start measurement processing of biological information by the measurement instrument 100, by performing an operation input of selecting the start button 3010. For each of the various functional buttons 3012, 3014 and 3016, for example, a function to start predetermined processing may be set.

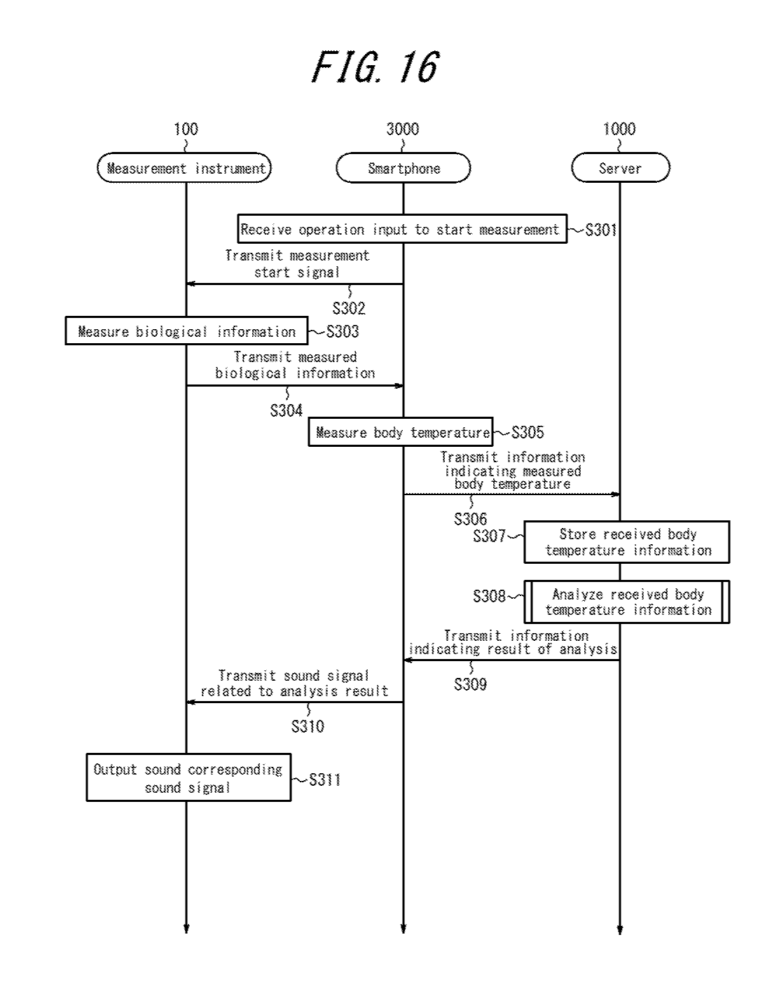

Next, an example of processing performed by the measurement system 20000 illustrated in FIG. 13 will be described with reference to FIG. 16. FIG. 16 is a sequence chart illustrating an example of processing by the measurement system 20000. The sequence illustrated in FIG. 16 may be started, for example, with a user wearing the measurement instrument 100.

Initially, the smartphone 3000 receives an operation input indicating measurement start from a subject (Step S301).

Upon receiving the operation input, the smartphone 3000 transmits a measurement start signal to the measurement instrument 100 (Step S302).

Based on the measurement start signal, the measurement instrument 100 measures biological information using the measurement unit 15 of the measurement apparatus 10 (Step S303).

Then, the measurement instrument 100 transmits the biological information measured by the measurement unit 15 to the smartphone 3000 (Step S304).

Based on the biological information acquired from the measurement instrument 100, the smartphone 3000 measures the subject's body temperature (Step S305).

Then, the smartphone 3000 transmits information indicating the measured body temperature to the server 1000 (Step S306).

Upon receiving the body temperature information from the smartphone 3000, the server 3000 stores the received body temperature information in the memory 1003 (Step S307).

Further, the server 1000 analyzes the body temperature information received from the smartphone 3000 (Step S308)

Then, the server 1000 transmits information indicating the result of the analysis to the smartphone 3000 (Step S309).

Based on the received analysis result, the smartphone 3000 transmits a sound signal related to the analysis result to the measurement instrument 100 (Step S310).

Then, the measurement instrument 100 causes the vibrator 16 to vibrate in accordance with the sound signal to transmit sound to the subject, thereby outputting a sound corresponding to the sound signal (Step S311). Through the output of the sound, the subject can be notified of the analysis result.

In Step S310, the smartphone 3000 may output a notification without transmitting the sound signal. For example, the smartphone 3000 may perform the notification by displaying the analysis result on the display 3034.

Further, for example, the controller 3031 of the smartphone 3000 may cause the display 3034 to display a list of music information stored in the memory 3032. In addition, the controller 3031 may cause the measurement instrument 100 to output music of the display list selected by a user via the input interface 3033.

However, the measurement instrument 100 according to the present disclosure is not limited to the examples described in the above embodiments. Note that the measurement instrument 100 may have various modifications including earphone type, such as a type in which on only one ear an earpiece is to be worn, or the like.

A plurality of embodiments has been described for a complete and clear disclosure. However, it is to be noted that the appended claims are not limited to the embodiments described above, and are to be construed as encompassing all of the possible modifications and alternate configurations that a person of ordinary skill in the art could make within the scope of the fundamental features illustrated in this disclosure. Moreover, each requirement described in these embodiments can be freely combined.

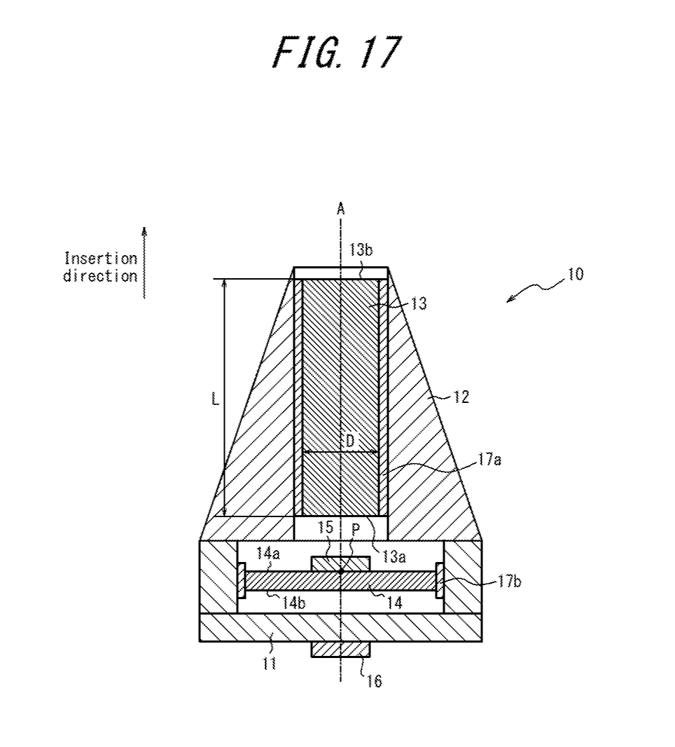

For example, the measurement apparatus 10 described with reference to FIG. 1 may further include vibration attenuation members. FIG. 17 is a schematic diagram illustrating the internal structure of a variation of the measurement apparatus 10. The measurement apparatus 10 illustrated in FIG. 17 includes vibration attenuation members 17a and 17b.

In the example illustrated in FIG. 17, the vibration attenuation member 17a is provided between the earpiece 12 and the metal tube 13. Further, the vibration attenuation member 17b is provided between the holder 11 and the substrate 14. The vibration attenuation member 17a and the vibration attenuation member 17b (hereinafter, also referred to as vibration attenuation members 17) are configured to reduce the effect of vibration caused by the vibrator 16 on members connected to the housing via the vibration attenuation members 17. In the example illustrated in FIG. 17, the members connected to the housing via the vibration attenuation members 17 are the metal tube 13 and the substrate 14. That is, in the example illustrated in FIG. 17, when the vibrator 16 vibrates, the holder 11, and the earpiece 12 coupled to the holder 11 vibrate. Through the vibration, sound is transmitted to the subject. On the other hand, since the metal tube 13 and the substrate 14 are connected to the housing via the vibration attenuation members 17, the vibration caused by the vibrator 16 would be absorbed by the attenuation members 17, and thus the effect of the vibration on the metal tube 13 and the substrate 14 would be reduced. That is, the metal tube 13 and the substrate 14 would not vibrate as much as the holder 11 and the earpiece 12. In this manner, by attenuating the vibration of the metal tube 13 and the substrate 14, the measurement unit 15 arranged on the substrate 14 can acquire infrared radiation in a more stable state, that is, in a state with less vibration. Thereby, the accuracy of body temperature detection, based on infrared radiation acquired by the measurement unit 15, can be improved.

The vibration attenuation members 17 may be formed from any member capable of attenuating vibration. For example, the vibration attenuation members 17 may be formed from at least one of rubber, urethane, and urethane foam. The material of the vibration attenuation members 17a provided between the earpiece 12 and the metal tube 13 can be selected by focusing on the attenuation characteristic of frequency in a frequency band close to the resonance frequency of the metal tube 13 so as to absorb the vibration of the earpiece 12 and the metal tube 13. The vibration attenuation member 17a is configured to reduce the transmission of vibration to the metal tube 13, in order to prevent the frequency of sound transmitted to the ear from being affected. Further, the vibration attenuation member 17b provided between the substrate 14 and the holder 11 is configured to attenuate the vibration of music so as not to be transmitted to the measurement unit 15 which serves as a sensor.

However, the vibration attenuation member 17b is not necessary to be provided between the holder 11 and the substrate 14. When the vibration attenuation member 17b is arranged between the measurement unit 15 and the vibrator 16, the measurement unit 15 can acquire infrared radiation in a more stable state. For example, the vibration attenuation member 17b may be arranged on the substrate 14 so as to surround the measurement unit 15.

1. A measurement apparatus, comprising:
a metal tube having a first end and a second end;
a measurement unit which is arranged at a first end side of the metal tube and is capable of measuring electromagnetic radiation incident from the second end of the metal tube;
a housing for holding the metal tube; and
a vibrator for vibrating the housing.

2. The measurement apparatus according to claim 1, wherein the vibrator is configured to vibrate the housing based on an applied sound signal.

3. The measurement apparatus according to claim 1, wherein the housing includes an earpiece to be inserted into an ear canal.

4. The measurement apparatus according to claim 1, wherein the electromagnetic radiation is infrared radiation.

5. The measurement apparatus according to claim 1, wherein the metal tube is a cylindrical member comprising an inner surface with high reflectance.

6. The measurement apparatus according to claim 1, wherein the vibrator includes a piezoelectric element.

7. The measurement apparatus according to claim 1, further comprising a vibration attenuation member which is arranged between the measurement unit and the vibrator to attenuate vibration caused by the vibrator.

8. The measurement apparatus according to claim 1, further comprising a vibration attenuation member which is arranged between the metal tube and the vibrator to attenuate vibration caused by the vibrator.

9. A measurement instrument, comprising:
a first measurement unit configured as a blood flow measurement apparatus capable of measuring blood flow; and
the measurement apparatus according to claim 1.

10. A measurement system, comprising:
the measurement apparatus according to claim 1;
a control apparatus for controlling operation of the measurement apparatus; and
a server connected to the measurement apparatus via a network, wherein the server is configured to receive information on electromagnetic radiation measured by the measurement apparatus, analyze the received information based on information stored in a memory, and transmit the analyzed information to the control apparatus.

11. The measurement system according to claim 10, wherein the server is configured to store at least one of an average body temperature of a group to which a user belongs and an individual average body temperature of a user, and generate the analyzed information based on the received information and at least one of the average body temperature and the individual average body temperature.

12. The measurement system according to claim 10, wherein a memory of the control apparatus is configured to store, as sound output information to be outputted to a measurement instrument, at least one of health information indicating a health condition, music information related to music, and environmental information around the measurement apparatus, wherein the measurement instrument comprises a first measurement unit configured as a blood flow measurement apparatus capable of measuring blood flow and the measurement apparatus according to claim 1.

13. A measurement system, comprising:
the measurement apparatus according to claim 1;
a smartphone configured to control operation of the measurement apparatus; and
a server connected to the measurement apparatus via a network, wherein the server is configured to receive information on electromagnetic radiation measured by the measurement apparatus, analyze the received information based on information stored in a memory, and transmit analysis information indicating a result of the analysis to a control apparatus.

14. A server connected to a control apparatus for controlling operation of the measurement apparatus according to claim 1 via a network; wherein the server is configured to
receive information on electromagnetic radiation measured by the measurement apparatus,
analyze the received information based on information stored in a memory, and
transmit analysis information indicating a result of the analysis to the control apparatus.

15. An analysis method performed by a server connected to a control apparatus configured to control operation of the measurement apparatus according to claim 1 via a network, comprising:
receiving information on electromagnetic radiation measured by the measurement apparatus;
analyzing the received information based on information stored in a memory; and
transmitting analysis information indicating a result of the analysis to the control apparatus.

16. A non-transitory computer readable storage medium storing an analysis program which, when executed by a computer, causes the computer to
receive information on electromagnetic radiation measured by the measurement apparatus according to claim 1;
analyze the received information based on information stored in a memory; and
transmit analysis information indicating a result of the analysis to a control apparatus.

17. A data structure configured for use in a computer, comprising:
information on electromagnetic radiation measured by a measurement apparatus according to claim 1;
a user ID of a user of the measurement apparatus;
an average body temperature of a group to which the user belongs; and
an individual average body temperature of the user; wherein
the computer is used to determine whether information on electromagnetic radiation measured by the measurement apparatus is abnormal based on at least one of the average body temperature and the individual average body temperature.
